(19) United States
(12) Patent Application Publication
(10) Pub. No.: US 2006/0247668 A1
(43) Pub. Date: Nov. 2, 2006

(54) Surgical tool

(76) Inventor: Kee B. Park, Jackson, MO (US)

Correspondence Address: Stephen R. Greiner, Esquire;GREINER LAW OFFICES, P.C., Suite 110, 6701 Democracy Blvd., Bethesda, MD, 20817, US

(21) Appl. No.: 11/116,231

(22) Filed: Apr. 28, 2005

(51) Int. Cl. A61B 17/22

(52) U.S. Cl. 606/160

(57) ABSTRACT

A surgical tool including a pair of levers that are pivotally connected together so as to define a pair of opposed rearward portions that serve as handles and a pair of opposed forward portions. A pair of ring curettes is affixed to the forward portions such that, when the rearward portions are squeezed together, the ring curettes are driven apart to cut material from opposite sides of a cavity into which they are inserted.

### FIELD OF THE INVENTION

The present invention relates generally to surgical implements and, more particularly, to curettes.

### BACKGROUND OF THE INVENTION

Spinal cord injuries can be the result of many things including ruptured, prolapsed, or herniated intervertebral disks. These injuries occur when ligaments are damaged so that disk material (the nucleus pulposus) can extrude through adjacent ligaments and compress the spinal cord. For younger individuals, disk herniation can occur when lifting a heavy load whereas sneezing can cause disk herniation in the elderly. Regardless of the cause, disk herniation can be painful.

Common symptoms of disk herniation can include: stiff necks, back aches, muscle spasms, pain while coughing or straining, and sensory disturbances in the arms and hands. Most disk herniation is treated conservatively, at least initially.

Conservative treatment consists of strict bed rest on a firm mattress and the use of drugs. Local heat applications are also employed, but prolonged heat applications increase the likelihood of congestion. Corsets can provide additional support for an individual with a lumbar disk problem. A frequent satisfactory result of such treatment involves a herniated disk receding into an intervertebral space on its own.

When the conservative approach is ineffective, surgery may be required. Surgical treatment of a herniated intervertebral disk consists of a laminectomy, which may or may not be followed with spinal fusion. A laminectomy is a procedure in which a portion of a vertebra is removed to expose the spinal cord. Then, a surgeon removes the portion of the nucleus pulposus that is protruding from the intervertebral disk toward the spinal cord. Spinal fusion, however, involves the removal of pieces of bone from another region of the body and grafting these pieces onto the vertebrae. Once the graft has “taken,” a firm bony bridge causes a permanent area of stiffness in the surrounding area that an individual must adjust to.

Due to the close proximity to the spinal cord, the removal of the nucleus pulposus is typically accomplished with handheld, unpowered implements. Despite the great promise of success for their patients, surgeons who perform many laminectomies often find the use of such implements to be personally debilitating. Repetitive cutting motions with conventional curettes can result in muscle aches and, in some cases, carpel tunnel syndrome. A need, therefore, exists for an improved cutting tool or curette for use during laminectomies and other surgical procedures that minimizes strain and the likelihood of repetitive motion injuries.

### SUMMARY OF THE INVENTION

In light of the problems associated with the performance of laminectomies and the cutting of the nucleus pulposus of intervertebral disks, it is a principal object of the invention to provide a hand-operated surgical tool that can cut through an intervertebral disk and other hard body parts without imparting great strain to a surgeon.

It is another object of the invention to provide a surgical tool of the type described that cuts by driving portions thereof away from one another thereby creating a pair of opposed cutting faces for the faster removal of tissues from an individual. This cutting configuration also tends to give the tool great stability in the hands of a surgeon with one cutting portion stabilizing the other.

It is a further object of the invention to provide a surgical tool of the type described whose cutting portions nest together for easy insertion into, and removal from, the tight spaces occupied by intervertebral disks.

It is an object of the invention to provide improved elements and arrangements thereof in a surgical tool for the purposes described that is lightweight in construction, inexpensive to manufacture, as well as dependable in use.

Briefly, the tool in accordance with this invention achieves the intended objects by featuring a pair of levers that are pivotally connected together so as to define a pair of opposed rearward portions that serve as handles and a pair of opposed forward portions. A ring curette is affixed to each the forward portions of the levers. Each ring curette is a circular band with a sharpened cutting edge that can be driven outwardly by squeezing the rearward portions of the levers toward one another. A spring mechanism is disposed between the rearward portions of the levers for normally biasing the ring curettes toward one another.

The foregoing and other objects, features and advantages of the present invention will become readily apparent upon further review of the following detailed description of the preferred embodiment as illustrated in the accompanying drawings.

### BRIEF DESCRIPTION OF THE DRAWINGS

The present invention may be more readily described with reference to the accompanying drawings, in which:

FIG. 1 is a side view of a surgical tool in accordance with the present invention.

FIG. 2 is a longitudinal cross-sectional view of the surgical tool of FIG. 1.

FIG. 3 is a bottom view of the surgical tool

FIG. 4 is a top view of the surgical tool.

FIG. 5 is a perspective view, from above, of the forward portions of the pivoting levers comprising the surgical tool.

FIG. 6 is a perspective view, from below, of the forward portions of the pivoting levers comprising the surgical tool.

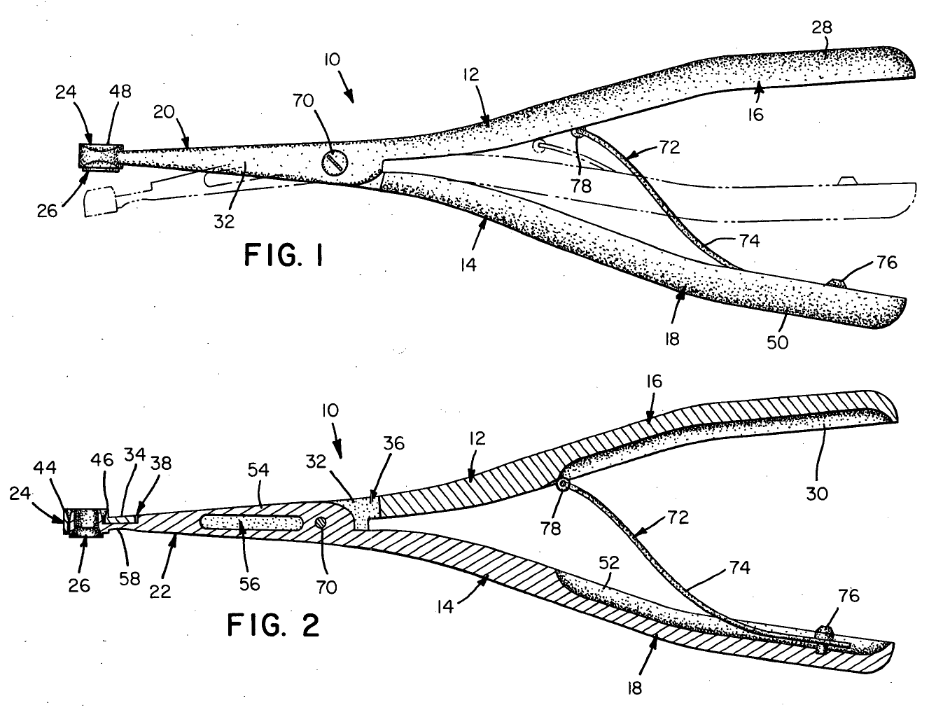

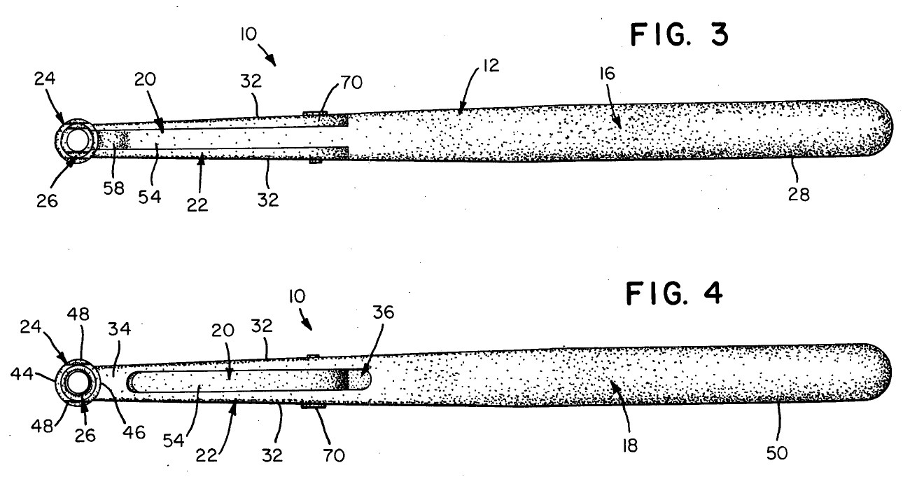

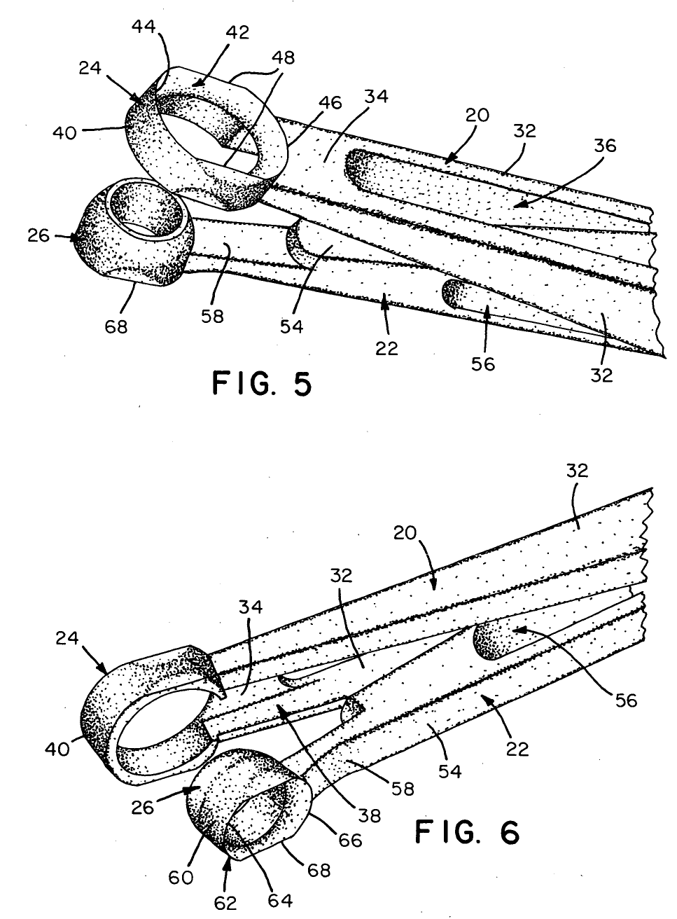

Similar reference characters denote corresponding features consistently throughout the accompanying drawings.

### DETAILED DESCRIPTION OF THE PREFERRED EMBODIMENT

Referring now to the FIGS., a surgical tool in accordance with the present invention is shown at 10. Tool 10 includes a pair of levers 12 and 14 that are pivotally connected together. Levers 12 and 14 are configured so that their rearward portions 16 and 18 serve as handles. The forward portions 20 and 22 of levers 12 and 14, however, carry ring curettes 24 and 26 that function as cutting implements. When rearward portions 16 and 18 are pivoted toward one another, curettes 24 and 26 are driven apart to cut material from opposite sides of a cavity into which such is inserted.

Rearward portion 16 and forward portion 20 of lever 12 are joined at a shallow angle. Rearward portion 16 comprises an elongated bar having a rounded, outer surface 28 for user comfort and a scalloped, inner surface 30 to reduce the weight of tool 10. Forward portion 20, however, includes a pair of elongated legs 32 that extend forwardly from rearward portion 16 in parallel fashion, tapering in height toward their free ends. Legs 32 are connected together at their free ends by a cross piece 34 so as to define a slot 36 bounded by rearward portion 16, legs 32 and cross piece 34. Cross piece 34, being relatively thin, extends partially from the outer surfaces of legs 32 toward the inner surfaces of legs 32 so that a notch 38 is formed between legs 32. Notch 38 opens inwardly to slot 36 and opens outwardly to ring curette 24.

Ring curette 24 includes a circular band 40 that is affixed to the free ends of legs 32 and to crosspiece 34 for reinforcement purposes. Band 40 has a diameter of about 0.5 inches (1.2 cm) which is somewhat greater than the distance between legs 32 and which dictates the maximum size of the cut that tool 10 can make. The height of band 40 is about 0.25 inches (0.58 cm), somewhat greater than the height of the free ends of legs 32, for maximum rigidity. The thickness of the band 40 is about 0.0625 inches (0.144 cm) so that a sharpened cutting edge 42 of great durability can be formed about the outer surface of band 40.

Sharpened cutting edge 42 is formed by milling the outer surface of band 40 so that is slopes inwardly toward the center of band 40. This milled surface has a plurality of distinct cutting regions or areas 44, 46 and 48 that serve to control the width of cuts made by curette 24 and prevent lateral gouging. As shown, forward area 44, farthest from legs 32, is semicircular in outline and concave relative to legs 32 as it opens toward them. Rearward area 46, immediately adjacent to legs 32 and crosspiece 34, is also semicircular in outline and convex relative to legs 32 as it opens away from them. A pair of lateral areas 48 connects the forward area 44 and rearward area 46 together and are positioned on opposite sides of tool 10. As shown, lateral areas 48 are linear for making cuts parallel to the longitudinal axis of tool 10.

Lever 14 has a rearward portion 18 and a forward portion 22 joined together at a shallow angle. Rearward portion 18 is an elongated bar having a rounded, outer surface 50 and a scalloped, inner surface 52. Forward portion 22, on the other hand, includes an elongated arm 54 sized for reciprocation within slot 36. Arm 54 includes a longitudinal cutout 56 to reduce weight and terminates at a finger 58 of reduced size that fits snugly within notch 38 and normally projects into circular band 40 of ring curette 24.

Ring curette 26 includes a circular band 60 affixed to the free end of finger 58. Band 60 has a diameter of about 0.1875 inches (0.43 cm) which is slightly smaller than the opening at the center of band 40 within which band 60 is normally positioned. The height of band 60 is about 0.3125 inches (0.72 cm) which is greater than that of band 40 so that the outer surface of band 60 projects from the inner surface of band 40 and the inner surface of band 60 is flush with the outer surface of band 40 when forward portions 20 and 22 are fully pivoted together. The thickness of the band 60 is about 0.0625 inches (0.144 cm).

A sharpened cutting edge 62 is formed by milling the outer surface of band 60 so that it slopes inwardly toward the center of band 60. Like cutting edge 42, cutting edge 62 has a plurality of cutting areas 64, 66 and 68. Forward area 64, farthest from finger 58, is semicircular in outline and concave relative to finger 58. Rearward area 66, adjoining finger 58, is also semicircular in outline and convex relative to finger 58. A pair of lateral areas 68 connect the forward area 64 and rearward area 66 and are positioned on opposite sides of tool 10. Lateral areas 68 are linear for making cuts that are parallel to the longitudinal axis of tool 10.

A pivot pin 70 connects levers 12 and 14 together. As shown, pivot pin 70 penetrates the inner ends of both legs 32 and arm 54. Pivot pin 70 is shown to be a screw so that tool 10 can be disassembled for easy cleaning, reconditioning and repair. Nonetheless, pin 70 can be formed from a rivet or any other suitable thing.

A spring mechanism 72 is provided between levers 12 and 14 to normally maintain ring curettes 24 and 26 in a nested relationship. Mechanism 72 includes a prong 74 formed of spring metal that is affixed at its rear end by means of a rivet 76 to the rear end of rearward portion 18 of lever 14. Prong 74 extends forwardly and inwardly toward the rearward portion 16 of lever 12. A friction-reducing roller 78, secured to the free end of prong 74, engages the inner surface 30 of rearward portion 16 and normally biases ring curettes 24 and 26 into a nested relationship.

Use of surgical tool 10 is straightforward. During a laminectomy, for example, a portion of a vertebra is removed to expose the spinal cord of a patient. Then, with tool 10 in hand, the surgeon removes a selected portion of the intervertebral disk that is protruding toward the spinal cord by slowly squeezing rearward portions 16 and 18 toward one another so as to drive ring curettes 24 and 26 into the intervertebral disk. After a cut has been made by curettes 24 and 26, spring mechanism 72 returns them to a nested position to dislodge the just cut disk material from curettes 24 and 26. The nested curettes 24 and 26 also permit the walls of the cavity formed in the intervertebral disk through the use of tool 10 to be more easily examined prior to the removal of additional disk material. After surgery has been performed, curettes 24 and 26 can be resharpened, if desired, and tool 10 can be sterilized for subsequent reuse.

While the invention has been described with a high degree of particularity, it will be appreciated by those skilled in the art that modifications can be made to it. For example, ring curettes 24 and 26 need not be circular in outline but, rather, can be provided with any suitable closed geometric shape. Therefore, it is to be understood that the present invention is not limited to the sole embodiment described above, but encompasses any and all embodiments within the scope of the following claims.

I claim:

1. A surgical tool, comprising:
a pair of levers being pivotally connected together so as to define a pair of opposed rearward portions that serve as handles and a pair of opposed forward portions;
a pair of ring curettes, each being respectively affixed to one of said forward portions whereby, when said rearward portions are squeezed together, said curettes are driven apart to cut material from opposite sides of a cavity into which such are inserted.

2. The surgical tool according to claim 1 further comprising a spring mechanism disposed between said rearward portions of said levers for normally biasing said ring curettes toward one another.

3. The surgical tool according to claim 1 wherein one of said ring curettes has a larger diameter than the other one of said ring curettes whereby said pair of ring curettes can compactly nest together with one being located within the other.

4. The surgical tool according to claim 4 wherein said sharpened cutting edge of each ring curette includes:
a forward cutting area being concave relative to said forward portions of said levers;
a rearward cutting area being convex relative to said forward portions of said levers; and,
a pair of lateral cutting areas connecting said forward cutting area to said rearward cutting area.

5. A surgical tool, comprising:
a pair of levers being pivotally connected together so as to define a pair of opposed rearward portions that serve as handles and a pair of opposed forward portions;
a pair of ring curettes, each being respectively affixed to one of said forward portions and each also including a circular band having a sharpened cutting edge that can be driven outwardly by squeezing said rearward portions of said levers together; and,
a spring mechanism disposed between said rearward portions of said levers for normally biasing said ring curettes toward one another.

6. The surgical tool according to claim 5 wherein one of said ring curettes has a larger diameter than the other one of said ring curettes so that said pair of ring curettes can nest together.

7. The surgical tool according to claim 5 wherein said sharpened cutting edge of each ring curette includes:
a forward cutting area being concave relative to said forward portions of said levers;
a rearward cutting area being convex relative to said forward portions of said levers; and,
a pair of lateral cutting areas connecting said forward cutting area to said rearward cutting area.

8. A surgical tool, comprising:
a pair of levers being pivotally connected together with each of said levers having a rearward portion and a forward portion;
one said forward portion including:
a pair of parallel legs extending from one said rearward portion to terminate at free ends; and,
a cross piece connecting said free ends of said legs together and defining an inwardly opening notch between said legs;
the other said forward portion including:
an arm extending from the other said rearward portion and being slidably positioning between said legs, said arm terminating at a finger that projects forwardly from said notch;
a first ring curette having a first diameter and being affixed to said parallel legs and said cross piece;
a second ring curette having a second diameter that is smaller than said first diameter and being affixed to said arm such that, when pivoted together, said second ring curette will nest within said first ring curette; and,
a spring mechanism disposed between said rearward portions of said levers for normally retaining said first ring curette and said second ring curette in a nested relationship.

9. The surgical tool according to claim 8 wherein said first ring curette and said second ring curette each include a sharpened cutting edge having:
a forward cutting area being concave relative to its associated forward portion;
a rearward cutting area being convex relative to its associated forward portion; and,
a pair of lateral cutting areas connecting said forward cutting area to said rearward cutting area.
